(19) United States
(12) Patent Application Publication
(10) Pub. No.: US 2024/0226110 A1
(43) Pub. Date: Jul. 11, 2024

(54) 5-HT2C AGONIST FOR USE IN TREATING CONDITIONS ASSOCIATED WITH CENTRAL HYPOVENTILATION

(71) Applicant: APNIMED, INC. (DELAWARE), Cambridge, MA (US)

(72) Inventors: Luigi TARANTO-MONTEMURRO, Brookline, MA (US); Ronald FARKAS, Clarsville, MD (US); Lawrence G. MILLER, Cambridge, MA (US)

(21) Appl. No.: 18/571,847

(22) PCT Filed: Jun. 29, 2022
(86) PCT No.: PCT/US2022/035548

§ 371 Date: Dec. 19, 2023

Related U.S. Application Data
(60) Provisional application No. 63/216,909, filed on Jun. 30, 2021.

(51) Int. Cl. A61K 31/55; A61K 31/433; A61P 11/16

(52) U.S. Cl. CPC A61K 31/55; A61K 31/433; A61P 11/16

(57) ABSTRACT

In general, the invention relates to pharmaceutical compositions comprising a 5-xHT2C agonist, and methods of treating conditions associated with central hypoventilation including administering a 5-HT2C agonist.

### CROSS-REFERENCE TO RELATED APPLICATION

This application claims priority to U.S. provisional application No. 63/216,909, filed Jun. 30, 2021, the entire contents of which are incorporated herein by reference.

### TECHNICAL FIELD

The present invention provides pharmaceutical compositions comprising a 5-HT2C agonist, and methods of treating conditions associated with central hypoventilation including administering a 5-HT2C agonist.

### BACKGROUND

Obesity hypoventilation syndrome (OHS) is a condition associated with obesity in which patients fail to breathe rapidly or deep enough, resulting in low oxygen levels and high blood CO₂ levels. Untreated OHS is associated with significant morbidity.

### SUMMARY

One aspect of the present invention provides a method of treating a subject having a condition associated with central hypoventilation, the method comprising administering to a subject in need thereof an effective amount of a 5-HT2C agonist.

Embodiments of this aspect of the invention may include one or more of the following optional features. In some embodiments the 5-HT2C agonist is selected from the group consisting of YM-348, lorcaserin, vabicaserin, Ro60-0175, MK-212, WAY161503, WAY163909, Org 37684, VER-3323, AL-38022A, LY448100, BVT.933, PNU-142633, Ro-60-0332, Org 12962, PNU-22394, PNU-181731, WAY-629, IK264, A-372159, CP-809101, fenfluarmine, lisuride, mesulergine (CU-32085), naphthylaminopropane (PAL-287), norfenfluramine, oxaflozane, PF-04479745, 8-chloro-2,3,4,5-tetrahydro-1H-3-benzazepine, 6-(2,2,2-trifluoroethylamino)-7-chloro-2,3,4,5-tetrahydro-1H-benzo[d]azepine, 1-(4-trifluoromethylbenzofur-7-yl)-3(S)-methylpiperazine (CPD-1; LS-193743), and 1-(4-trifluoromethylbenzofur-7-yl)-3(S)-ethylpiperazine, and their pharmaceutically acceptable salts or prodrugs. In some embodiments, the 5-HT2C agonist is lorcaserin or a pharmaceutically acceptable salt thereof. In some embodiments, the 5-HT2C agonist (e.g., lorcaserin or a salt or prodrug of lorcaserin) is in an immediate release formulation. In some embodiments, the 5-HT2C agonist (e.g., lorcaserin or a salt or prodrug of lorcaserin) is in a delayed release formulation. In some embodiments, the single composition is an oral administration form (e.g., a syrup, slurry, pill, tablet, troche, capsule, or patch). In some embodiments, the lorcaserin is administered once daily at a dose of from about 5 mg to about 100 mg or from about 10 mg to about 60 mg. In some embodiments, the method further comprises administering to the subject in need thereof an effective amount of a carbonic anhydrase inhibitor or a pharmaceutically acceptable salt thereof. In some embodiments, the carbonic anhydrase inhibitor is acetazolamide or a pharmaceutically acceptable salt thereof.

In some embodiments, the condition associated with central hypoventilation is obesity hypoventilation syndrome (OHS), congenital central hypoventilation syndrome, chronic obstructive pulmonary disease (COPD), or COPD with hypercapnia. In some embodiments, the condition associated with central hypoventilation is OHS. In some embodiments, the 5-HT2C agonist is in an immediate release formulation and is administered at a dosage of from about 5 to about 100 mg. In some embodiments, the subject is being treated or has been treated with continuous positive airway pressure (CPAP). In some embodiments, the subject is being treated or has been treated with non-invasive ventilation (NIV).

Another aspect of the present invention provides a pharmaceutical composition comprising a 5-HT2C agonist and a pharmaceutically acceptable excipient or carrier.

Embodiments of this aspect of the invention may include one or more of the following optional features. In some embodiments the 5-HT2C agonist is selected from the group consisting of YM-348, lorcaserin, vabicaserin, Ro60-0175, MK-212, WAY161503, WAY163909, Org 37684, VER-3323, AL-38022A, LY448100, BVT.933, PNU-142633, Ro-60-0332, Org 12962, PNU-22394, PNU-181731, WAY-629, IK264, A-372159, CP-809101, fenfluarmine, lisuride, mesulergine (CU-32085), naphthylaminopropane (PAL-287), norfenfluramine, oxaflozane, PF-04479745, 8-chloro-2,3,4,5-tetrahydro-1H-3-benzazepine, 6-(2,2,2-trifluoroethylamino)-7-chloro-2,3,4,5-tetrahydro-1H-benzo[d]azepine, 1-(4-trifluoromethylbenzofur-7-yl)-3(S)-methylpiperazine (CPD-1; LS-193743), and 1-(4-trifluoromethylbenzofur-7-yl)-3(S)-ethylpiperazine, or a pharmaceutically acceptable salt thereof. In some embodiments, the 5-HT2C agonist is lorcaserin or a pharmaceutically acceptable salt thereof. In some embodiments, the pharmaceutical composition further comprises a carbonic anhydrase inhibitor. In some embodiments, the carbonic anhydrase inhibitor is acetazolamide or a pharmaceutically acceptable salt thereof. In some embodiments, the pharmaceutical composition is for use in treating a condition associated with central hypoventilation is obesity hypoventilation syndrome (OHS), congenital central hypoventilation syndrome, chronic obstructive pulmonary disease (COPD), or COPD with hypercapnia. In some embodiments, the condition associated with central hypoventilation is obesity hypoventilation syndrome (OHS).

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

The following figures are provided by way of example and are not intended to limit the scope of the claimed invention.

FIG. 1. Comparison of lorcaserin and placebo effect for various ventilatory parameters (A-D) reflecting the upper airway collapsibility and muscle activity.

FIG. 2. Ventilation during NREM sleep of a patient on placebo (top, A) and on lorcaserin (bottom, B). Despite the presence of hypopneas in both conditions, ventilation increased substantially during lorcaserin night.

### DETAILED DESCRIPTION

Obesity hypoventilation syndrome (OHS), also known as Pickwickian syndrome, is a combination of obesity (body mass index≥30 kg/m²), daytime hypercapnia (arterial carbon dioxide tension≥45 mmHg) and sleep-disordered breathing, after ruling out other disorders that may cause alveolar hypoventilation. Most patients are severely obese (BMI>40 kg/m²). These patients do not increase their ventilatory response sufficiently to maintain normal blood CO₂ levels, and often have reduced oxygen saturation both in wakefulness and during sleep.

The epidemiology of OHS is only partially characterized, with estimates from 0.15-0.4% of the adult population in the US but there is little definitive data.

Most, but not all, patients have some degree of sleep apnea, often severe, and continuous positive airway pressure (CPAP) is generally the first-line therapy. However, not all patients respond to CPAP, leaving a group with limited treatment options. Non-invasive ventilation (NIV) may be used in these patients, especially in more severe cases not responding to CPAP.

Lorcaserin is a proposed respiratory stimulant, which appears to act at 5-HT2C receptors in the brainstem. These receptors are involved, together with 5-HT2A receptors, in the increase of respiratory drive in response to the rising in CO₂ during sleep in OSA patients.

Serotonin 2C (“5-HT2C”) receptors also modulate the excitability and responsiveness of the HMN, as suggested by data collected in an animal model showing that the stimulation of the serotonin (5-HT) 2C receptor with the 5-HT2A agonist 2,5-dimethoxy-4-iodoaminophentamine (DOI) increased genioglossus activity and reduced upper airway collapsibility in anesthetized rats. These receptors are present in the pre-motor areas of the hypoglossal motor nucleus (HMN) and comprise the primary hypoglossal afferents including among others the Kölliker-Fuse region, trigeminal nuclei and central tegmental field.

No pharmacologic treatments of OHS have been explored or approved to date.

### Methods of Treatment

The methods described herein include methods for the treatment of conditions associated with central hypoventilation. In some embodiments, the condition is obesity hypoventilation syndrome (OHS), congenital central hypoventilation syndrome, chronic obstructive pulmonary disease (COPD), or COPD with hypercapnia.

Generally, the methods include administering a therapeutically effective amount of a serotonin 2C receptor agonist (“5-HT2C agonist”) to a subject who is in need of, or who has been determined to be in need of, such treatment. In some embodiments, the 5-HT2C agonist is selected from the group consisting of YM-348, lorcaserin, vabicaserin, Ro60-0175, MK-212, WAY161503, WAY163909, Org 37684, VER-3323, AL-38022A, LY448100, BVT.933, PNU-142633, Ro-60-0332, Org 12962, PNU-22394, PNU-181731, WAY-629, IK264, A-372159, CP-809101, fenfluarmine, lisuride, mesulergine (CU-32085), naphthylaminopropane (PAL-287), norfenfluramine, oxaflozane, PF-04479745, 8-chloro-2,3,4,5-tetrahydro-1H-3-benzazepine, 6-(2,2,2-trifluoroethylamino)-7-chloro-2,3,4,5-tetrahydro-1H-benzo[d]azepine, 1-(4-trifluoromethylbenzofur-7-yl)-3(S)-methylpiperazine (CPD-1; LS-193743), and 1-(4-trifluoromethylbenzofur-7-yl)-3(S)-ethylpiperazine, and their pharmaceutically acceptable salts or prodrugs. In some embodiments, the 5-HT2C agonist is lorcaserin or a pharmaceutically acceptable salt thereof. In some embodiments, the 5-HT2C agonist (e.g., lorcaserin or a salt or prodrug of lorcaserin) is administered as a monotherapy.

In some embodiments, the 5-HT2C agonist (e.g., lorcaserin or a salt or prodrug of lorcaserin) is administered in combination with one or more additional active agents (e.g., two or more, or three or more, additional active agents). In some embodiments, the one or more additional active agents is a carbonic anhydrase inhibitor. Carbonic anhydrase inhibitors include, but are not limited to, acetazolamide, methazolamide, dorzolamide, and brinzolamide. In some embodiments, the carbonic anhydrase inhibitor is acetazolamide or a pharmaceutically acceptable thereof.

As used in this context, to “treat” means to ameliorate at least one symptom of the disorder associated with central hypoventilation. In some embodiments, a disorder associated with central hypoventilation is obesity hypoventilation syndrome (OHS). Often, OHS, results in sleepiness, lack of energy, breathlessness, headache, and depression during the daytime. At nighttime, OHS results in loud and frequent snoring during sleep and/or breathing pauses. OHS patients can also have right heart failure with lower extremity edema. Thus, a treatment can result in reduction of snoring, apneas, breathing pauses, breathlessness, headache, and other symptoms associated with OHS.

In general, an “effective amount” of a compound refers to an amount sufficient to elicit the desired biological response, e.g., to treat a condition associated with central hypoventilation e.g., to treat obesity hypoventilation syndrome (OHS).

Patients with a “hypoventilation syndromes” generally have mild hypercarbia or elevated serum bicarbonate levels when awake, which sometimes worsen during sleep. Hypoventilation syndromes include, and are not limited to, congenital central hypoventilation syndrome (CCHS) and obesity hypoventilation syndrome (OHS).

“Hypoventilation” is defined as elevated levels of arterial carbon dioxide (pCO₂), e.g., elevated by at least 10 mm Hg above the upper limit of normal. Treatment of hypoventilation syndromes is generally aimed at correcting or improving the waking pCO₂.

An effective amount can be administered in one or more administrations, applications or dosages. The compositions can be administered from one or more times per day to one or more times per week; including once every other day. In some embodiments, the compositions are administered daily. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent. In some embodiments, the therapeutically effective amount encompasses an amount that normalizes or improves waking pCO₂ levels.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, “pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salts” includes “pharmaceutically acceptable acid addition salts” and “pharmaceutically acceptable base addition salts.” “Pharmaceutically acceptable acid addition salts” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salts” include those derived from inorganic bases such as sodium, potassium, lithium, ammonium, calcium, magnesium, iron, zinc, copper, manganese, aluminum salts, and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines, and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.)

As used herein, the term “unit dosage form” is defined to refer to the form in which the compound is administered to a subject. Specifically, the unit dosage form can be, for example, a pill, capsule, or tablet. In some embodiments, the unit dosage form is a capsule.

As used herein, “solid dosage form” means a pharmaceutical dose(s) in solid form, e.g., tablets, capsules, granules, powders, sachets, reconstitutable powders, dry powder inhalers and chewables.

For the compounds disclosed herein, single stereochemical isomers, as well as enantiomers, diastereomers, cis/trans conformation isomers, and rotational isomers, and racemic and non-racemic mixtures thereof, are within the scope of the invention. Unless otherwise indicated, all tautomeric forms of the compounds disclosed herein are within the scope of the invention.

Lorcaserin is the generic name of the pharmaceutical substance with the chemical name (1R)-8-chloro-1-methyl-2,3,4,5-tetrahydro-1H-3-benzazepine, and stereoisomers, hydrates and salts thereof. Lorcaserin is commercially available as lorcaserin hydrochloride (Belviq®). In some embodiments, lorcaserin may be lorcaserin hydrochloride.

Acetazolamide is the generic name of the pharmaceutical substance with the chemical name N-(5-Sulfamoyl-1,3,4-thiadiazol-2-yl)acetamide. Acetazolamide is available as a generic medication as well as sold under the trade names Diamox, Dacarb, and others.

In some embodiments, the methods include administering a dose of from about 5 mg to about 100 mg of a 5-HT2C agonist (e.g., lorcaserin or a salt or prodrug of lorcaserin). In some embodiments, the methods include administering a dose of from about 10 mg to about 60 mg of a 5-HT2C agonist (e.g., lorcaserin or a salt or prodrug of lorcaserin). In some embodiments, the methods include administering a dose of from about 20 mg to about 40 mg of a 5-HT2C agonist (e.g., lorcaserin or a salt or prodrug of lorcaserin). In some embodiments, the methods include administering a dose of about 5, 10, 15, 20, 25, 30, 35, 40, 45, 50, 60, 70, 80, 90, or 100 mg of 5-HT2C agonist (e.g., lorcaserin or a salt or prodrug of lorcaserin). In some embodiments, the dose is a daily dose. In some embodiments, the dose is a twice-daily dose.

In some embodiments, the methods include administering a dose of from about 5 mg to about 20 mg of lorcaserin or a pharmaceutically acceptable salt thereof. In some embodiments, the methods include administering a dose of from about 20 mg to about 40 mg of lorcaserin or a pharmaceutically acceptable salt thereof (e.g., 20 mg, 25 mg, 30 mg, 35 mg, or 40 mg).

Provided herein is a method of treating a subject having a condition associated with central hypoventilation, the method comprising administering to a subject in need thereof an effective amount of 5-HT2C agonist (e.g., lorcaserin) or a pharmaceutically acceptable salt thereof. In some embodiments, the condition associated with central hypoventilation is obesity hypoventilation syndrome (OHS), congenital central hypoventilation syndrome, chronic obstructive pulmonary disease (COPD), or COPD with hypercapnia. In some embodiments, the condition is obesity hypoventilation syndrome (OHS). In some embodiments, the subject is being treated or has been treated with continuous positive airway pressure (CPAP). In some embodiments, the subject has been treated with CPAP. In some embodiments, the subject is not responsive to CPAP therapy. In some embodiments, the subject is being treated or has been treated with non-invasive ventilation (NIV). In some embodiments, the subject has been treated with NIV. In some embodiments, the subject is not responsive to NIV.

In some embodiments, the condition associated with central hypoventilation is congenital central hypoventilation syndrome (CCHS). CCHS is a disorder wherein control of breathing is absent or impaired. A CCHS child's respiratory response to low blood oxygen saturation (hypoxia) or to CO₂ retention (hypercapnia) is typically sluggish during awake hours and absent, or reduced during sleep, serious illness, and/or stress.

In some embodiments, the condition associated with central hypoventilation is chronic obstructive pulmonary disease (COPD) or COPD with hypercapnia. COPD is a chronic inflammatory lung disease that causes obstructed airflow from the lungs. Symptoms include breathing difficulty, cough, mucus (sputum) production and wheezing. Hypercapnia, also known as hypercarbia and CO₂ retention, is a condition of abnormally elevated carbon dioxide levels in the blood. Hypercapnia is normally caused by hypoventilation of the body leading to CO₂ retention. Hypercapnia is the elevation in the partial pressure of carbon dioxide (PaCO₂) above 45 mm Hg on Arterial Blood Gas readings. Hypercapnia can eventually cause hypoxaemia due to reduced respiratory drive.

Also provided herein is a method of treating a subject having a condition associated with central hypoventilation (e.g., obesity hypoventilation syndrome (OHS)), the method comprising administering to a subject in need thereof a once daily dose of from about 10 mg to about 60 mg of lorcaserin or a pharmaceutically acceptable salt thereof. In some embodiments, the method comprises administering to a subject in need thereof a twice daily dose of from about 10 mg to about 60 mg of lorcaserin or a pharmaceutically acceptable salt thereof. In some embodiments, the method comprises administering to a subject in need thereof a once daily dose of from about 20 mg to about 40 mg of lorcaserin or a pharmaceutically acceptable salt thereof. In some embodiments, the method comprises administering to a subject in need thereof a twice daily dose of from about 20 mg to about 40 mg of lorcaserin or a pharmaceutically acceptable salt thereof.

In some embodiments, the 5-HT2C agonist (e.g., lorcaserin or a salt or prodrug of lorcaserin) may be in an immediate release formulation. In some embodiments, the 5-HT2C agonist (e.g., lorcaserin or a salt or prodrug of lorcaserin) may be in an extended release formulation.

In some embodiments the 5-HT2C agonist is selected from the group consisting of, but not limited to, YM-348, lorcaserin, vabicaserin, Ro60-0175, MK-212, WAY161503, WAY163909, Org 37684, VER-3323, AL-38022A, LY448100, BVT.933, PNU-142633, Ro-60-0332, Org 12962, PNU-22394, PNU-181731, WAY-629, IK264, A-372159, CP-809101, fenfluarmine, lisuride, mesulergine (CU-32085), naphthylaminopropane (PAL-287), norfenfluramine, oxaflozane, PF-04479745, 8-chloro-2,3,4,5-tetrahydro-1H-3-benzazepine, 6-(2,2,2-trifluoroethylamino)-7-chloro-2,3,4,5-tetrahydro-1H-benzo[d]azepine, 1-(4-trifluoromethylbenzofur-7-yl)-3(S)-methylpiperazine (CPD-1; LS-193743), and 1-(4-trifluoromethylbenzofur-7-yl)-3(S)-ethylpiperazine, and their pharmaceutically acceptable salts or prodrugs.

In some embodiments, the 5-HT2C agonist is lorcaserin or a pharmaceutically acceptable salt thereof.

Structures of 5-HT2C agonists are provided in the following table.

| | |
|---|---|
| YM-348 | 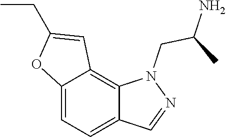 [SMILES: CCc1cc2c(ccc3cnn(C[C@H](C)N)c32)o1] |
| Ro60-0175 | 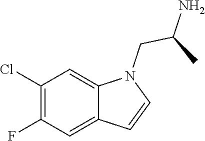 |
| MK-212 | 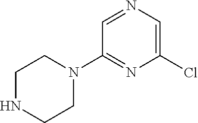 [SMILES: Clc1cncc(N2CCNCC2)n1] |
| WAY161503 | 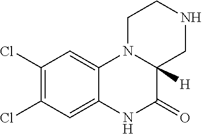 [SMILES: O=C1Nc2cc(Cl)c(Cl)cc2N2CCNC[C@H]12] |
| WAY163909 | 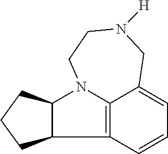 [SMILES: c1cc2c3c(c1)[C@H]1CCC[C@H]1N3CCNC2] |
| Org 37684 | 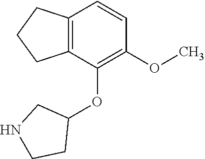 [SMILES: COc1ccc2c(c1OC1CCNC1)CCC2] |
| VER-3323 | 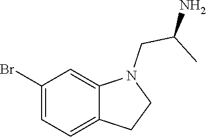 |
| AL-38022A | 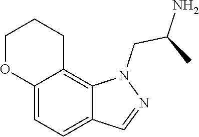 [SMILES: C[C@H](N)Cn1ncc2ccc3c(c21)CCCO3] |
| LY448100 | 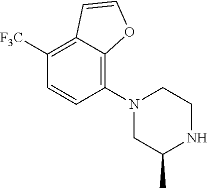 [SMILES: C[C@H]1CN(c2ccc(C(F)(F)F)c3ccoc23)CCN1] |
| BVT.933 | 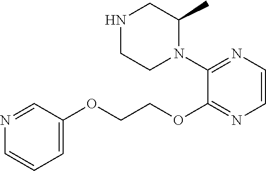 [SMILES: C[C@@H]1CNCCN1c1nccnc1OCCOc1cccnc1] |
| PNU-142633 | 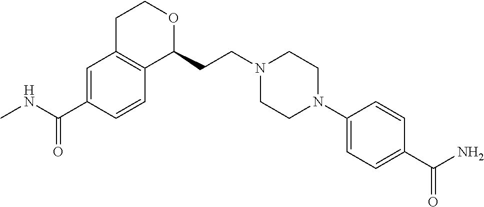 [SMILES: CNC(=O)c1ccc2c(c1)CCO[C@H]2CCN1CCN(c2ccc(C(N)=O)cc2)CC1] |
| Ro-60-0332 | 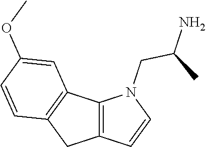 [SMILES: COc1ccc2c(c1)-c1c(ccn1C[C@H](C)N)C2] |
| Org 12962 | 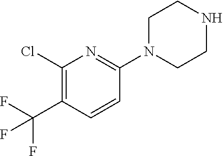 [SMILES: FC(F)(F)c1ccc(N2CCNCC2)nc1Cl] |
| PNU-22394 | 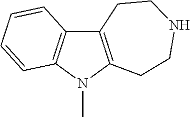 |
| PNU-181731 | 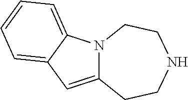 [SMILES: c1ccc2c(c1)cc1n2CCNCC1] |
| WAY-629 | 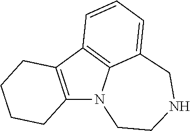 [SMILES: c1cc2c3c(c1)c1c(n3CCNC2)CCCC1] |
| IK264 | 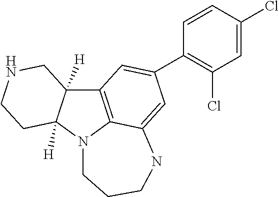 [SMILES: Clc1ccc(-c2cc3c4c(c2)[C@@H]2CNCC[C@@H]2N4CCCN3)c(Cl)c1] |
| A-372159 | 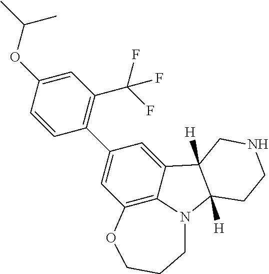 [SMILES: CC(C)Oc1ccc(-c2cc3c4c(c2)[C@@H]2CNCC[C@@H]2N4CCCO3)c(C(F)(F)F)c1] |
| CP-809101 | 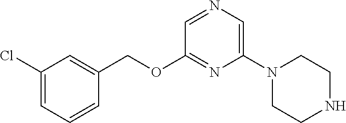 [SMILES: Clc1cccc(COc2cncc(N3CCNCC3)n2)c1] |
| PF-04479745 | 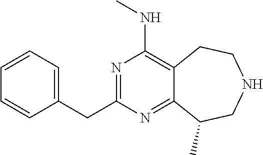 [SMILES: CNc1nc(Cc2ccccc2)nc2c1CCNC[C@@H]2C] |

### Pharmaceutical Compositions

The methods described herein include the use of pharmaceutical compositions comprising a 5-HT2C agonist. In some embodiments the 5-HT2C agonist is selected from the group consisting of, but not limited to, YM-348, lorcaserin, vabicaserin, Ro60-0175, MK-212, WAY161503, WAY163909, Org 37684, VER-3323, AL-38022A, LY448100, BVT.933, PNU-142633, Ro-60-0332, Org 12962, PNU-22394, PNU-181731, WAY-629, IK264, A-372159, CP-809101, fenfluarmine, lisuride, mesulergine (CU-32085), naphthylaminopropane (PAL-287), norfenfluramine, oxaflozane, PF-04479745, 8-chloro-2,3,4,5-tetrahydro-1H-3-benzazepine, 6-(2,2,2-trifluoroethylamino)-7-chloro-2,3,4,5-tetrahydro-1H-benzo[d]azepine, 1-(4-trifluoromethylbenzofur-7-yl)-3(S)-methylpiperazine (CPD-1; LS-193743), and 1-(4-trifluoromethylbenzofur-7-yl)-3(S)-ethylpiperazine, and their pharmaceutically acceptable salts or prodrugs. In some embodiments, the 5-HT2C agonist is lorcaserin or a pharmaceutically acceptable salt thereof. In some embodiments, the lorcaserin or pharmaceutically acceptable salt thereof is present in the pharmaceutical composition in an amount of from about 5 mg to about 100 mg, or from about 10 mg to about 60 mg, or from about 20 mg to about 40 mg, e.g., 20 mg, 25 mg, 30 mg, 35 mg, or 40 mg.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Supplementary active compounds can also be incorporated into the compositions, e.g., carbonic anhydrase inhibitors, e.g., acetazolamide, or a pharmaceutically acceptable salt thereof. In some embodiments, the pharmaceutical composition comprises a 5-HT2C agonist (e.g., lorcaserin, or a salt or prodrug of lorcaserin) and a carbonic anhydrase inhibitor. In some embodiments, the method of treating a subject having a condition associated with central hypoventilation comprises administering to a subject in need thereof an effective amount of (i) a 5-HT2C agonist (e.g., lorcaserin, or a salt or prodrug of lorcaserin), and (ii) a carbonic anhydrase inhibitor. Carbonic anhydrase inhibitors include, but are not limited to, acetazolamide, methazolamide, dorzolamide, and brinzolamide.

In some embodiments, lorcaserin or a pharmaceutically acceptable salt thereof is in the form of lorcaserin hydrochloride.

Pharmaceutical compositions are typically formulated to be compatible with its intended route of administration. Examples of routes of administration include systemic oral, transdermal administration, and parenteral administration.

Methods of formulating suitable pharmaceutical compositions using pharmaceutically acceptable carriers are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, oral compositions generally include an inert diluent or an edible carrier. For the purpose of oral therapeutic administration, the active compound(s) can be incorporated with excipients and used in the form of pills, tablets, troches, or capsules, e.g., gelatin capsules.

Oral compositions can also be prepared using a fluid carrier. In some embodiments, a composition according to the present invention may be a unit dosage form. In some embodiments, a composition according to the present invention may be a solid dosage form, e.g., a tablet or capsule.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In some embodiments, a 5-HT2C agonist (e.g., lorcaserin, or a salt or prodrug of lorcaserin) and one or more additional active agents are disposed together in one or more pharmaceutically acceptable carriers to form a single dosage agent. The single dosage agent comprises the 5-HT2C agonist (e.g., lorcaserin, or a salt or prodrug of lorcaserin), the one or more additional active agents, and the pharmaceutically acceptable carrier. In other embodiments, 5-HT2C agonist (e.g., lorcaserin, or a salt or prodrug of lorcaserin) and one or more additional active agents are separately disposed in one or more pharmaceutically acceptable carriers to form separate dosing agents. The separate dosing agents may be used in any combination to provide treatment to a subject having a condition associated with central hypoventilation is obesity hypoventilation syndrome (OHS), congenital central hypoventilation syndrome, chronic obstructive pulmonary disease (COPD), or COPD with hypercapnia.

Systemic administration of a 5-HT2C agonist (e.g., lorcaserin, or a salt or prodrug of lorcaserin) can also be by transdermal means, e.g., using a patch, gel, or lotion, to be applied to the skin. For transdermal administration, penetrants appropriate to the permeation of the epidermal barrier can be used in the formulation. Such penetrants are generally known in the art. For example, for transdermal administration, the active compounds can formulated into ointments, salves, gels, or creams as generally known in the art. The gel and/or lotion can be provided in individual sachets, or via a metered-dose pump that is applied daily; see, e.g., Cohn, J A. et al., An update on the use of transdermal oxybutynin in the management of overactive bladder disorder, Ther Adv Urol. 2016, April; 8(2): 83-90.

In one embodiment, the therapeutic compounds are prepared with carriers that will protect the therapeutic compounds against rapid elimination from the body, such as a controlled release formulation, including implants and microencapsulated delivery systems.

Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

Administration of a 5-HT2C agonist (e.g., lorcaserin, or a salt or prodrug of lorcaserin) can also be by parenteral, e.g., intravenous, means.

The pharmaceutical compositions can be included in a container, pack, or dispenser together with instructions for administration or use in a method described herein.

In some embodiments, the pharmaceutical composition is for use in treating a condition associated with central hypoventilation. In some embodiments, the condition is obesity hypoventilation syndrome (OHS). In some embodiments, the condition is congenital central hypoventilation syndrome (CCHS), chronic obstructive pulmonary disease (COPD), or COPD with hypercapnia.

In certain embodiments, provided herein is a pharmaceutical composition comprising lorcaserin or a pharmaceutically acceptable salt thereof for use in treating obesity hypoventilation syndrome (OHS).

In some embodiments, provided herein is a pharmaceutical composition comprising lorcaserin or a pharmaceutically acceptable salt thereof and optionally acetazolamide or a pharmaceutically acceptable salt thereof for use in treating COPD with hypercapnia. In some embodiments, provided herein is a pharmaceutical composition comprising lorcaserin or a pharmaceutically acceptable salt thereof and acetazolamide or a pharmaceutically acceptable salt thereof for use in treating COPD with hypercapnia.

### EXAMPLES

The invention is further described in the following examples, which do not limit the scope of the invention described in the claims.

### Example 1. Crossover Pilot Study

### Background

Obesity hypoventilation syndrome (OHS) is defined as a combination of obesity (body mass index≥30 kg/m²), daytime hypercapnia (arterial carbon dioxide tension≥45 mmHg) and sleep-disordered breathing, after ruling out other disorders that may cause alveolar hypoventilation. Most patients are severely obese (BMI>40 kg/m²). These patients do not increase their ventilatory response sufficiently to maintain normal blood CO₂ levels, and often have reduced oxygen saturation both in wakefulness and during sleep. The epidemiology of OHS is only partially characterized, with estimates from 0.15-0.4% of the adult population in the US but little definitive data.

Most, but not all, patients have some degree of sleep apnea, often severe, and CPAP is generally the first-line therapy. However, not all patients respond to CPAP, leaving a group with limited treatment options. Non-invasive ventilation (NIV) may be used in these patients, especially in more severe cases not responding to CPAP. There are scattered case reports involving use of respiratory stimulants, e.g., acetazolamide, but such treatments have not been attempted broadly.

Lorcaserin is a proposed respiratory stimulant, which appears to act at 5-HT2C receptors in the brainstem. These receptors are involved, together with 5-HT2A receptors, in the increase of respiratory drive in response to the rising in CO₂ during sleep. Serotonin 2C (“5-HT2C”) receptors also modulate the excitability and responsiveness of the HMN, as suggested by data collected in an animal model showing that the stimulation of the serotonin (5-HT) 2C receptor with the 5-HT2A agonist 2,5-dimethoxy-4-iodoaminophentamine (DOI) increased genioglossus activity and reduced upper airway collapsibility in anesthetized rats. These receptors are present in the pre-motor areas of the hypoglossal motor nucleus (HMN) and comprise the primary hypoglossal afferents including among others the Kolliker-Fuse region, trigeminal nuclei and central tegmental field.

### Method

In a crossover pilot trial including 6 OSA patients, the patients were administered in random order lorcaserin 15 mg or placebo before bedtime for one night.

Patients were outfitted with the usual polysomnography equipment plus a pneumotach and oronasal mask for accurate ventilation measurements. The effect of lorcaserin on several ventilatory parameters reflecting the upper airway collapsibility and muscle activity were measured using validated algorithms.

### Results

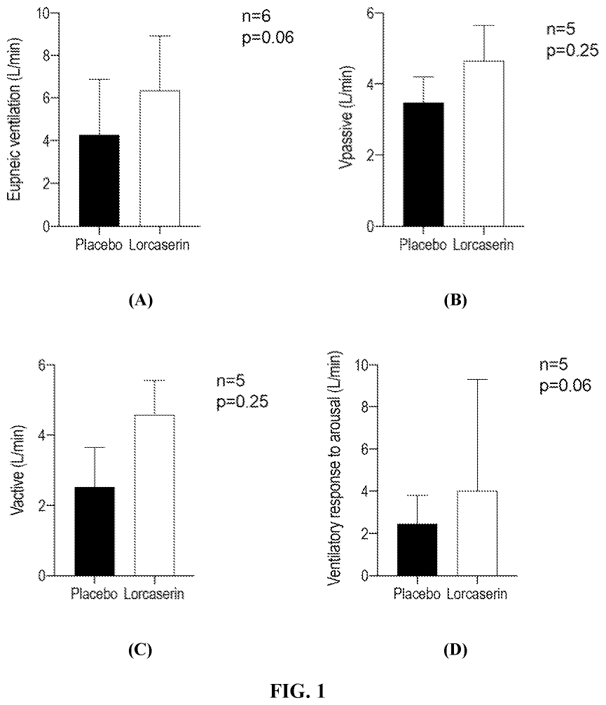

The study showed that lorcaserin increased overall ventilation during sleep compared to placebo (median [IQR] increase: 1.85 [0.15; 3.86] L/min, p=0.06), indicating a more patent upper airway (FIG. 1). Lorcaserin also significantly increased by 2.3 [0; 2.9]L/min (p=0.25) the ventilation at the arousal threshold when the pharyngeal muscles are maximally active (Vactive). These data, taken together, suggest that lorcaserin has an important primary effect on ventilatory drive, and (secondarily) on upper airway patency and muscle recruitment.

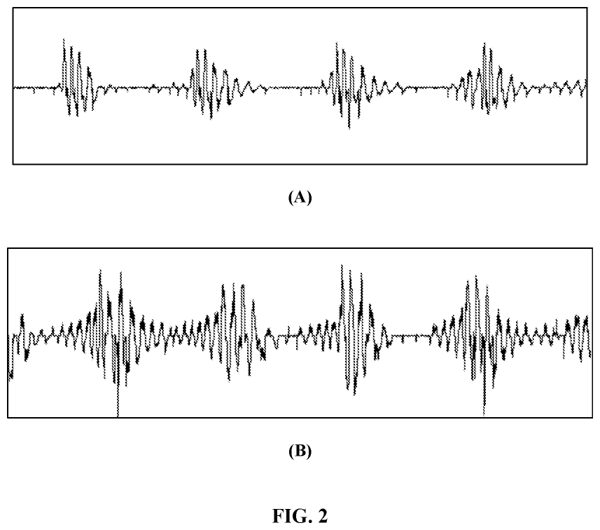

In patients with a collapsible upper airway, large variations in ventilatory drive can increase the propensity for repetitive pharyngeal obstructions and destabilize breathing in two ways. First, the periods of low drive can reduce pharyngeal muscle activation, making the airway floppier and more susceptible to collapse despite low suction pressures. Second, the periods of high drive can produce negative inspiratory pressures that overcome airway dilating forces and suck the airway closed. Either extreme may be a problem (FIG. 2).

FIG. 2 shows ventilation during NREM sleep of a patient on placebo (top, A) and on lorcaserin (bottom, B). Despite the presence of hypopneas in both conditions, ventilation increased substantially during lorcaserin night. In conclusion, the overall effect of lorcaserin shows utility in a number of disorders characterized primarily by hypoventilation.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject having a condition associated with central hypoventilation, the method comprising administering to a subject in need thereof an effective amount of a 5-HT2C agonist or a pharmaceutically acceptable salt thereof or prodrug thereof.

2. The method of claim 1, wherein the 5-HT2C agonist is selected from the group consisting of YM-348, lorcaserin, vabicaserin, Ro60-0175, MK-212, WAY161503, WAY163909, Org 37684, VER-3323, AL-38022A, LY448100, BVT.933, PNU-142633, Ro-60-0332, Org 12962, PNU-22394, PNU-181731, WAY-629, IK264, A-372159, CP-809101, fenfluarmine, lisuride, mesulergine (CU-32085), naphthylaminopropane (PAL-287), norfenfluramine, oxaflozane, PF-04479745, 8-chloro-2,3,4,5-tetrahydro-1H-3-benzazepine, 6-(2,2,2-trifluoroethylamino)-7-chloro-2,3,4,5-tetrahydro-1H-benzo[d]azepine, 1-(4-trifluoromethylbenzofur-7-yl)-3(S)-methylpiperazine (CPD-1; LS-193743), and 1-(4-trifluoromethylbenzofur-7-yl)-3(S)-ethylpiperazine, or a pharmaceutically acceptable salt thereof.

3. The method of claim 1 or 2, comprising administering to the subject a daily dose of from about 5 mg to about 100 mg of the 5-HT2C agonist.

4. The method of any one of claims 1-3, comprising administering to the subject a daily dose of from about 10 mg to about 60 mg of the 5-HT2C agonist.

5. The method of any one of claims 1-4, wherein the 5-HT2C agonist is administered orally.

6. The method of any one of claims 1-5, wherein the 5-HT2C agonist is in an immediate release formulation.

7. The method of any one of claims 1-5, wherein the 5-HT2C agonist is in an extended release formulation.

8. The method of claim 1 or 5, wherein the 5-HT2C agonist is in an immediate release formulation and is administered at a dosage of from about 5 to about 100 mg.

9. The method of claim 1 or 5, wherein the 5-HT2C agonist is in an extended release formulation and is administered at a dosage of from about 5 to about 100 mg.

10. The method of any one of claims 1-9, wherein the method further comprises administering to the subject in need thereof an effective amount of a carbonic anhydrase inhibitor or a pharmaceutically acceptable salt thereof.

11. The method of claim 10, wherein the carbonic anhydrase inhibitor is acetazolamide or a pharmaceutically acceptable salt thereof.

12. The method of any one of claims 1-9, wherein the method is a monotherapy of the 5-HT2C agonist.

13. The method of any one of claims 1-12, wherein the 5-HT2C agonist is lorcaserin or a pharmaceutically acceptable salt thereof.

14. The method of any one of claims 1-13, wherein the condition associated with central hypoventilation is obesity hypoventilation syndrome (OHS), congenital central hypoventilation syndrome, chronic obstructive pulmonary disease (COPD), or COPD with hypercapnia.

15. The method of any one of claims 1-13, wherein the condition associated with central hypoventilation is obesity hypoventilation syndrome (OHS).

16. The method of any one of claims 1-15, wherein the subject is being treated or has been treated with continuous positive airway pressure (CPAP).

17. The method of any one of claims 1-15, wherein the subject is being treated or has been treated with non-invasive ventilation (NIV).

18. The method of any one of claims 1-17, wherein the lorcaserin or pharmaceutically acceptable salt thereof is administered at a dose of from about 10 mg to about 60 mg once daily.

19. The method of claim any one of claims 1-18 wherein the 5-HT2C agonist is administered orally in an oral administration form selected from a syrup, slurry, pill, tablet, troche, capsule, or patch.

20. A 5-HT2C agonist for use in treating a subject having a condition associated with central hypoventilation.

21. The use of claim 20, wherein the 5-HT2C agonist is selected from the group consisting of YM-348, lorcaserin, vabicaserin, Ro60-0175, MK-212, WAY161503, WAY163909, Org 37684, VER-3323, AL-38022A, LY448100, BVT.933, PNU-142633, Ro-60-0332, Org 12962, PNU-22394, PNU-181731, WAY-629, IK264, A-372159, CP-809101, fenfluarmine, lisuride, mesulergine (CU-32085), naphthylaminopropane (PAL-287), norfenfluramine, oxaflozane, PF-04479745, 8-chloro-2,3,4,5-tetrahydro-1H-3-benzazepine, 6-(2,2,2-trifluoroethylamino)-7-chloro-2,3,4,5-tetrahydro-1H-benzo[d]azepine, 1-(4-trifluoromethylbenzofur-7-yl)-3(S)-methylpiperazine (CPD-1; LS-193743), and 1-(4-trifluoromethylbenzofur-7-yl)-3(S)-ethylpiperazine, or a pharmaceutically acceptable salt thereof.

22. The use of claim 20 or 21, wherein the 5-HT2C agonist is lorcaserin or a pharmaceutically acceptable salt thereof.

23. The use of any one of claims 20-22, wherein the condition associated with central hypoventilation is obesity hypoventilation syndrome (OHS), congenital central hypoventilation syndrome, chronic obstructive pulmonary disease (COPD), or COPD with hypercapnia.

24. The use of any one of claims 20-23, wherein the condition associated with central hypoventilation is obesity hypoventilation syndrome (OHS).

25. A pharmaceutical composition comprising a 5-HT2C agonist, and optionally a pharmaceutically acceptable carrier, for use in treating a subject having a condition associated with central hypoventilation.

26. The pharmaceutical composition for use of claim 25, wherein the 5-HT2C agonist is selected from the group consisting of YM-348, lorcaserin, vabicaserin, Ro60-0175, MK-212, WAY161503, WAY163909, Org 37684, VER-3323, AL-38022A, LY448100, BVT.933, PNU-142633, Ro-60-0332, Org 12962, PNU-22394, PNU-181731, WAY-629, IK264, A-372159, CP-809101, fenfluarmine, lisuride, mesulergine (CU-32085), naphthylaminopropane (PAL-287), norfenfluramine, oxaflozane, PF-04479745, 8-chloro-2,3,4,5-tetrahydro-1H-3-benzazepine, 6-(2,2,2-trifluoroethylamino)-7-chloro-2,3,4,5-tetrahydro-1H-benzo[d]azepine, 1-(4-trifluoromethylbenzofur-7-yl)-3(S)-methylpiperazine (CPD-1; LS-193743), and 1-(4-trifluoromethylbenzofur-7-yl)-3(S)-ethylpiperazine, or a pharmaceutically acceptable salt thereof.

27. The pharmaceutical composition of claim 25 or 26, wherein the 5-HT2C agonist is lorcaserin or a pharmaceutically acceptable salt thereof.

28. The pharmaceutical composition of any one of claims 25-27, further comprising a carbonic anhydrase inhibitor.

29. The pharmaceutical composition of claim 28, wherein the carbonic anhydrase inhibitor is acetazolamide or a pharmaceutically acceptable salt thereof.

30. The pharmaceutical composition of any one of claims 25-29, wherein the condition associated with central hypoventilation is obesity hypoventilation syndrome (OHS), congenital central hypoventilation syndrome, chronic obstructive pulmonary disease (COPD), or COPD with hypercapnia.

31. The pharmaceutical composition of any one of claims 25-29, wherein the condition associated with central hypoventilation is obesity hypoventilation syndrome (OHS).
